(19) United States
(12) Patent Application Publication
(10) Pub. No.: US 2024/0000890 A1
(43) Pub. Date: Jan. 4, 2024

(54) NEURAL REGENERATION WITH SYNTHETIC PROTEIN ADMINISTRATION

(71) Applicant: Retinal Solutions, LLC, Rochester, MI (US)

(72) Inventors: Kimberly Drenser, Rochester, MI (US); Michael T. Trese, Rochester, MI (US); Antonio Capone, Rochester, MI (US)

(73) Assignee: Retinal Solutions, LLC, Rochester, MI (US)

(21) Appl. No.: 17/791,960

(22) PCT Filed: Jan. 11, 2021
(86) PCT No.: PCT/US2021/012895

§ 371 Date: Jul. 11, 2022

Related U.S. Application Data
(60) Provisional application No. 62/958,925, filed on Jan. 9, 2020.

(51) Int. Cl. A61K 38/18

(52) U.S. Cl. CPC A61K 38/18

(57) ABSTRACT

A method for neural regeneration is provided at specific situses that include the inner ear and retina, where Ganglion cells respond to the method through at least stimulation of such cells. As a result, the method provides for reversing clinical conditions associated with the nerve degradation or disease. Specific clinical conditions reversed at least in part through nerve regeneration include hearing loss, tinnitus, and a host of neurotrophic retinopathies, diabetes, Norrie disease, and others. Nerve regeneration is accomplished with a protein that is a truncated synthetic polypeptide related to native norrin protein. Truncated norrin proteins have a longer half-life in the situs than native norrin proteins. A version of the truncated norrin protein lacks a cleavage site for a subject protease enzyme that cleaves native norrin proteins and thereby shortens the useful life of the therapeutic protein.

### CROSS REFERENCE TO RELATED APPLICATIONS

This application claims priority benefit of U.S. Provisional Application Ser. No. 62/958,925 filed 9 Jan. 2020, the contents of which are hereby incorporated by reference.

### FIELD OF THE INVENTION

The present invention is directed generally to methods of nerve regeneration in the retina, middle ear, or inner ear anatomies; and in particular, the use of synthetic norrin-like protein to stimulate neurogenesis therein.

### BACKGROUND OF THE INVENTION

Alterations in norrin function are associated with many pediatric vitreoretinopathies, such as Norrie disease (ND),¹⁻³ familial exudative vitreoretinopathy (FEVR),⁴⁻⁵ Coats disease,⁶ and retinopathy of prematurity (ROP).⁷⁻⁹ A unifying characteristic in these diseases is an aberration of ear and retinal vascular development, demonstrating varying degrees of peripheral avascular retina, abnormal vascularization with retinal neovascularization (NV), subretinal exudation, abnormal vascularization and degeneration in the stria vascularis and cochlea, abnormal vascularization and degeneration in other parts of the ear, and hair loss in the organ of Corti.¹⁰

At the cellular level, it is widely accepted that disruption of Norrin-Fzd-4 signaling is the key causative factor. Frizzled-4 is one of 11 Frizzled transmembrane receptors known to participate in Wnt signaling. Inside the cell, the Wnt signal can activate three pathways: one canonical (Wnt/β-Catenin) and two noncanonical (Wnt/PCP and Wnt/Ca). There is evidence that norrin may activate all three of these intracellular Wnt pathways.¹⁰⁻¹⁴ The Norrin-Fzd-4 signaling cascade plays a central role in the development and maintenance of the inner ear and retinal vasculature.¹⁰

Norrin is a small secreted protein with a cysteine-knot motif.^{10,15,16} The cysteine-knot motif is highly conserved in many growth factors including transforming growth factor-β (TGF-β), human chorionic gonadotropin, nerve growth factor, and platelet-derived growth factor. Norrin serves as a ligand for the Frizzled receptor subtype 4 (Fz4). Norrin binds Fz4 with nanomolar affinity (Xu, et al., Cell, 2004; 116:883-895; Clevers. Curr Biol, 2004; 14:R436-437; Nichrs, Dev Cell, 2004; 6:453-454). Norrin interaction with Fz4 is dependent on the cell surface receptors LRP5 and LRP6. (Xu, 2004). Norrin also promotes neuron protection through cell surface receptor LRP1. Frizzled receptors are coupled to the Wnt/β-catenin canonical signaling pathway. Norrin activates the Wnt/β-catenin canonical signaling pathway through Fz4 and LRP5 or LRP6 by binding specifically and with affinity.¹⁰ LGR4, LGR5, and LGR6 are transmembrane cell receptors also known to mediate Wnt signaling. (Deng et al., Journal of cell Science, 2013, 126: 2060-2068). Norrin binds to Fz4, LGR4, LGR5, and LGR6, activating the Wnt/β-catenin canonical signaling pathway, and promoting neurogenesis. The inactivation of glycogen synthase kinase (GSK) 3β and Axin through frizzled receptor binding stabilizes β-catenin, which subsequently accumulates in the cell nucleus and activates the transduction of target genes that are crucial in the G1-S-phase transition, such as cyclin D1 or c-Myc. (Willert et al., Curr Opin Genet Dev. 1998; 8:95-102). Suppression of norrin activity has been shown to preclude angiogenesis associated with ocular disease (US 2010/0129375). Suppression of norrin activity has also been shown to cause aberrant vascularization and hair cell loss in the inner ear associated with hearing loss.¹⁰ Fz4 is expressed in the inner ear including in the organ of Corti, inner and outer hair cells, and the stria vascularis. Evidence shows that Fz4 is necessary for inner ear maintenance and/or survival.¹⁰

LGR4 is expressed in proliferating cells of diverse tissues, including adult stem cells and progenitor cells. (Deng et al., 2013). LGR5 is an adult stem cell marker. (Chen et al., Aging Cell, 2015, 1-9). LGR5 cells are generated at late stages of retinal and inner ear development. In the retina, LGR5 cells exhibit properties of differentiated amacrine interneurons. (Cheg et al., 2015). Nevertheless, LGR5 amacrine cells contribute to regeneration of new retinal cells in the adult stage. The generation of new retinal cells, including retinal neurons and Müller glia from LGR5 amacrine cells, begins in early adulthood and continues as the animal ages. (Cheng et al., 2015). Given that LGR5 cells function as adult stem cells in multiple tissues and organs, this evidence implies that mammalian nerve cells are capable of regeneration and LGR5 cells function as an endogenous nerve regenerative source. Norrin is known to stimulate Wnt-signaling via Fz4, LGR4, LGR5, and LGR6 binding (as well as via LRP5 and LRP6 binding). As there are examples in the animal kingdom of organisms that can reconnect severed nerves and even regrow a damaged eye, pathways likely exist in humans to the do the same, even if they are not normally active.

The structural similarity of norrin to other growth factors suggests that norrin may have a function in addition to traditional Wnt-signaling, despite the fact that norrin is best characterized as a Wnt receptor ligand. This theory is supported by norrin's lack of structural similarity to that of other Wnt proteins.¹⁷ A previous study demonstrated that endogenous expression of norrin inhibits oxygen-induced retinopathy (OIR) in a mouse model.¹⁸ However, the half-life of naturally occurring wild versions of norrin are extremely short and are not effective for use in capillary stabilization and vascular regeneration in retinal tissue.

Thus, there exists a need for methods of nerve regeneration in the ear and the retina for treating, mitigating, preventing, and/or reversing the effects of nerve damage and nerve degeneration in the ear and the retina illustratively including hearing and vision loss. The present invention is directed to these, as well as other, important needs in the art.

### SUMMARY OF THE INVENTION

A method is provided for nerve regeneration in a living subject at a situs in a need thereof. The method includes administering to the situs an effective amount of an N-terminus norrin truncate that has a polypeptide N-terminus cleavage relative to a native norrin protein of up to 40 amino acid residues retaining a cysteine-knot motif of the native norrin protein and capable of binding to nerve cells in the situs, or a norrin mutant having at least 85% amino acid identity to SEQ ID NO. 1 and retaining a cysteine-knot motif of the native norrin protein and capable of binding to the nerve cells in the situs. After sufficient time, the N-terminus norrin truncate or the norrin mutant selectively up-regulates gene expression of at least one of FZD4, LGR4, LGR5, LGR6, or combinations thereof, and activates a Wnt signaling pathway, to stimulate neurogenesis of the nerve cells at the situs of the living subject. The method has implications in reversing hearing loss and improving visual acuity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and other aspects of the present invention will be elucidated in the accompanying drawings and following detailed description of the invention.

FIG. 1 is a photograph illustrating vascular structure (shown in red) and nerves (shown in green), and are readily seen tracking each other towards a destination;

FIGS. 2A-2E illustrate stereotyped axon and vessel navigation where overlapping guidance cues direct growth of both the nerves and blood vessels;

FIGS. 3A-3C illustrate the process of norrin angiogenesis through the Fzd4 receptor on endothelial cells, where FIG. 3A illustrates a canonical pathway, FIG. 3B shows a non-canonical or planar cell polarity pathway, and FIG. 3C shows a Wnt-Ca²⁺ pathway;

FIG. 4A illustrates norrin-mediated growth of endothelial buds;

FIG. 4B illustrates norrin-mediated growth of endothelial buds of FIG. 4A in an anatomical context with vascular structure (shown in red) and nerves (shown in green);

FIG. 5 illustrates norrin mediated FZD4 and LGR4 activation on retinal ganglion cells;

FIG. 6 is a bar graph illustrating norrin gene expression in embroids indicating that norrin plays a role in neurogenesis;

FIGS. 7A-7D are a series of four photographs illustrating an neurofilament-L (NFL) immunostain (green) of differentiated embroid bodies (2 weeks) depicting two control photos as shown and two norrin truncate-mutant treated photos, where FIG. 7A is a first control and FIG. 7B is the corresponding norrin treated photo, and FIG. 7C is a second control and FIG. 7D is the corresponding norrin treated photo;

FIGS. 8A-8D are a series of four photographs illustrating a nestin immunostain (green) of differentiated embroid bodies (2 weeks) depicting two control photos and two norrin truncate-mutant treated photos; where FIG. 8A is a first control and FIG. 8B is the corresponding norrin treated photo, and FIG. 8C is a first control and FIG. 8D is the corresponding norrin treated photo;

FIGS. 9A-9I are a series of six photographs and three graphs illustrating an increase in proliferation of retinal progenitor cells and retinal thickness in mice with ectopic expression of norrin;

FIG. 10 is a bar graph illustrating the effect of norrin truncate-mutant treatment on retinal ganglion cells (RGC) where the norrin treatment significantly increases mature retinal ganglion cells and the effect is sustained;

FIG. 11 is a bar graph illustrating the effect of norrin truncate-mutant treatment on RGC cell density;

FIGS. 12A-12D are a series of photographs taken at 30 minutes, 2 hours, 6 hours, and 24 hours that illustrate the effect of norrin truncate-mutant on RGC5 showing that norrin promotes dendrite/axon growth and cell survival, increases nuclear β-catenin, increases neurites, and increases intercellular communication;

FIGS. 13A-13C illustrate that norrin truncate-mutant increases nuclear β-catenin (FIG. 13A), norrin's role in activating Wnt-signaling and stimulating nerve regeneration (FIG. 13B), and promotes cell survival (FIG. 13C);

FIGS. 14A-14C illustrate the effect of norrin truncate-mutant on RGC5 as evidenced by protein expression levels (FIG. 14A), tPA and uPA levels (FIG. 14B), and the associated cell viability (FIG. 14C);

FIG. 15 illustrates a proposed mechanism for norrin promotion of neuron protection through LRP1 in accordance with embodiments of the invention;

FIGS. 16A-16D are a series of four photographs illustrating a control sample panel (FIG. 16A), an NMDA panel (FIG. 16B), an NMDA-Wnt3 panel (FIG. 16C), and an NMDA-Norrin panel (FIG. 16D) where the red immunostain is a glutamine synthetase (Müller cell) marker, and the blue immunostain is a nuclear stain;

FIG. 17 is a photograph illustrating a co-immunostain of Chx10 and Pax6 which are specific markers for retinal progenitor cells and is a co-immunostain commonly observed with norrin treatment following NMDA injury;

FIG. 18 is a photograph illustrating a side population of hematopoetic stem cells-GFP cells introduced with intravitreal injection and an intravitreal injection of norrin after 7 days;

FIG. 19 is a graph illustrating that norrin increases the number of Brn3a-labeled RGCs in OIR eyes; and

FIGS. 20A and 20B are two photographs illustrating LGR4 immunostained in the RGCs, the inner edge of the amacrine cells, and the outer plexiform layer, respectively.

### DETAILED DESCRIPTION OF THE INVENTION

The present invention has utility as a method for neural regeneration. Specific situses of nerve regeneration according to the present invention include the inner ear and retina. Ganglion cells respond to an inventive method through at least stimulation of such cells. As a result, the present invention offers the prospect of reversing clinical conditions associated with the nerve degradation or disease. Specific clinical conditions reversed at least in part through nerve regeneration include hearing loss, tinnitus, and a host of neurotrphic retinopathies, diabetes, Norrie disease, and others detailed herein.

Inventive embodiments of the disclosed method provide nerve regeneration with a protein that is a truncated synthetic polypeptide related to native norrin protein. Embodiments of the truncated norrin protein have a longer half-life in the situs than native norrin proteins. A preferred version of the truncated norrin protein lacks a cleavage site for a subject protease enzyme that cleaves native norrin proteins and thereby shortens the useful life of the therapeutic protein.

Embodiments of the inventive method encourage neurogenesis are demonstrated with exogenous treatment by truncated norrin in oxygen-induced retinopathy (OIR) mice. The therapeutic feasibility of intravitreal injection and intra-ear injection of the norrin protein and its effect on retinal and inner ear development, respectively, by activating Wnt-signaling is also shown.

The following definitions are used herein with respect to the understanding of the present invention.

“Administering” is defined herein as a way of providing a therapeutic protein or polypeptide, or a composition containing the same to a subject situs in need thereof for nerve regeneration. Such an administration can be by any route including, without limitation, oral, transdermal (e.g., oral mucosa), by injection (e.g., subcutaneous, intravenous, parenterally, intraperitoneally, intratympanic, intraocular), by inhalation (e.g., oral or nasal), or topical (e.g., eyedrops, eardrops, cream, etc.). Pharmaceutical preparations are, of course, given by forms suitable for each administration route.

By “alteration” is meant a change (increase or decrease) in the expression levels or activity of a gene or polypeptide as detected by standard art known methods such as those described herein. As used herein, an alteration includes at least a 10% change in expression levels, preferably a 25% change, more preferably a 40% change, and most preferably a 50% or greater change in expression levels.

By “analog” it is meant a molecule that is not identical, but has analogous functional or structural features to a norrin protein. For example, a polypeptide analog retains the biological activity of a corresponding naturally-occurring norrin, while having certain biochemical modifications that enhance the analog's function relative to a naturally occurring polypeptide. By way of non-limiting example, such biochemical modifications could increase the analog's protease resistance, solubility, membrane permeability, or half-life, without altering, for example, ligand binding. An analog may include an unnatural amino acid.

In this disclosure. “comprises,” “comprising.” “containing” and “having” and the like can have the meaning ascribed to them in U.S. Patent law and can mean “includes,” “including,” and the like and the term is open-ended, allowing for the presence of more than that which is recited so long as basic or novel characteristics of that which is recited is not changed by the presence of more than that which is recited, but excludes prior art embodiments; “consisting essentially of” or “consists essentially” likewise have the meaning ascribed in U.S. Patent law.

By “control” is meant a standard or reference status.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means that specifically includes late-phase angiographic posterior and peripheral vascular leakage (LAPPEL). For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a native norrin. This portion contains, preferably, at least 40%, 50%, 60%, 70%, 80%, or 90% of the entire length of the 133 amino acid residues of the native human norrin polypeptide. A fragment may contain 40, 50, 60, 70, 80, 90, 100, 110, 120, 130 or even up to 132 amino acid residues thereof.

By “truncate” is meant to include a fragment of norrin that has a polypeptide terminus cleavage of the norrin protein of up 40 amino acid residues.

By an “isolated polypeptide” is meant a polypeptide analog of norrin that has been separated from components that naturally accompany it. Typically, the polypeptide is isolated when it is at least 60%, by weight, free from proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

Norrin is meant to define a polypeptide or fragment thereof having at least about 85% amino acid identity to NCBI Accession No. NP_000257.1, as shown below, and having the ability to bind the frizzled-4 receptor, the LGR4, LGR5, and LGR6 receptors, or combinations thereof, of ear and retinal nerve cells, illustratively including retinal ganglion cells, the auditory nerve, and spiral ganglion cells.

(SEQ ID NO. 1)
gi14557789lreflNP_000257.11 norrin precursor
[Homo sapiens]
MRKHVLAASFSMLSLLVIMGDTDSKTDSSFIMDSDPRRCMRH
HYVDSISHPLYKCSSKMVLLARCEGHCSQASRSEPLVSFSTV
LKQPFRSSCHCCRPQTSKLKALRLRCSGGMRLTATYRYILSC
HCEECNS

As used herein, “obtaining” as in “obtaining an agent” includes synthesizing, purchasing, or otherwise acquiring the agent.

The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

Sequence identity is typically measured using sequence analysis software (for example, Sequence Analysis Software Package of the Genetics Computer Group. University of Wisconsin Biotechnology Center, 1710 University Avenue, Madison, Wis. 53705, BLAST, BESTFIT. GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e′³ and e″¹⁰⁰ indicating a closely related sequence.

As used herein, the terms “treat.” “treated,” “treating,” “treatment,” and the like refer to reducing or ameliorating a disorder and/or symptoms associated with nerve damage, nerve degeneration, and aberrant nerve generation.

Typically, a therapeutically effective dosage should produce a serum concentration of compound of from about 0.1 ng/ml to 100 μg/ml.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%. 6%, 5%, 4%, 3%, 2%, 1%, 0.5%, 0.1%, 0.05%, or 0.01% of the stated value. Unless otherwise clear from context, all numerical values provided herein are modified by the term about.

Norrin is a 133 amino acid long protein that is secreted into the extracellular space. Two primary domains define the general norrin protein structure: a signal peptide directs localization of the molecule; and a cysteine-knot motif provides the tertiary confirmation required for frizzled-4 receptor binding. (Meitinger, T, et al, Nat Genet, 1993; 5:376-380; Berger, W, et al. Hum Mol Genet, 1996; 5:51-59). Truncates and fragments of norrin that retain the ability to bind frizzled-4, LGR4, LGR5, LGR6 receptor, or combinations thereof, are operative herein. In some inventive embodiments a truncate or fragment of norrin retains the cysteine-knot motif.

The importance of the cysteine knot-motif is highlighted by computer modeling that demonstrates the requirement of disulfide bonds between the cysteine residues in forming the structural confirmation of norrin. However, mutations in regions other than the cysteine knot-motif produce incomplete protein folding and result in familial exudative vitreoretinopathy (FEVR), related vitreoretinopathies, malformation of structures in the inner ear illustratively including the organ of Corti and the stria vascularis, and inner ear hair loss which can cause hearing impairment or even hearing loss.

In certain inventive embodiments there exists a −24 residue N-terminus truncate of norrin, with the following amino acid sequence:

(SEQ ID NO. 2)
KTDSSFIMDSDPRRCMRHHYVDSISHPLYKCSSKMVLLAR
CEGHCSQASRSEPLVSFSTVLKQPFRSSCHCCRPQTSKLK
ALRLRCSGGMRLTATYRYILSCHCEECNS
(Accession # Q00604)

It has been found that some fragments and truncations such as SEQ ID NO: 2 have improved solubility compared to norrin.

The invention further embraces variants and equivalents which are substantially homologous to norrin and still retain the ability to selectively bind the frizzled-4, LGR4, LGR5, and LGR6 receptors, or combinations thereof. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid.

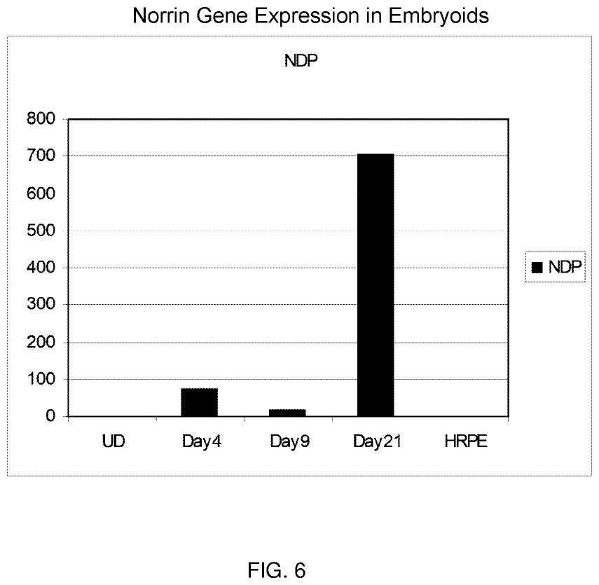

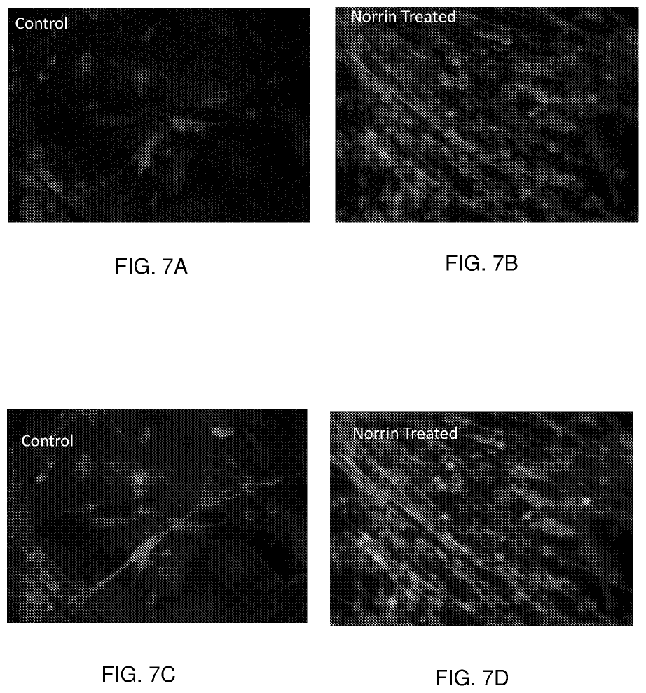

The norrin of the present invention is a synthetic norrin retaining binding properties to frizzled-4, LGR4, LGR5, LGR6, or combinations thereof. It is appreciated that the synthetic norrin of the present invention selectively up-regulates gene expression of at least one of FZD4, LGR4, LGR5, LGR6, or combinations thereof. It is further appreciated that the synthetic norrin of the present invention binds to at least one of FZD4, LGR4, LGR5, LGR6, or combinations thereof, activating the Wnt signaling pathway, thereby stimulating nerve regeneration in the ear and the retina. It will be recognized in the art that some amino acid sequences of the invention can be varied without significant effect of the structure or function of the protein. Thus, the invention further includes variations of the norrin which show substantial activity; such mutants include deletions, insertions, inversions, repeats, and type substitutions. Norrin mutants operable herein illustratively include amino acid substitutions relative to SEQ ID NO: 1 of R64E. Optionally the biologically active peptide is a multiple mutant relative to SEQ ID NO: 1: R64E: MRKHVLAASFSMLSLLVIMGDTDSKTDSSFIMDSDPRRCMRHHYVDSISHPLYKCSS KMVLLAECEGHCSQASRSEPLVSFSTVLKQPFRSSCHCCRPQTSKLKALRLRCSGGM RLTATYRYILSCHCEECNS (SEQ ID NO. 3). Optionally the biologically active peptide is a multiple mutant relative to SEQ ID NO: 1: T26A: MRKHVLAASFSMLSLLVIMGDTDSKADSSFIMDSDPRRCMRHHYVDSISHPLYKCSS KMVLLARCEGHCSQASRSEPLVSFSTVLKQPFRSSCHCCRPQTSKLKALRLRCSCGGM RLTATYRYILSCHCEECNS (SEQ ID NO. 4), S28A: MRKHVLAASFSMLSLLVIMGDTDSKTDASFIMDSDPRRCMRHHYVDSISHPLYKCSS KMVLLARCEGHCSQASRSEPLVSFSTVLKQPFRSSCHCCRPQTSKLKALRLRCSGGM RLTATYRYILSCHCEECNS (SEQ ID NO. 5), S29A: MRKHVLAASFSMLSLLVIMGDTDSKTDSAFIMDSDPRRCMRHHYVDSISHPLYKCSS KMVLLARCEGHCSQASRSEPLVSFSTVLKQPFRSSCHCCRPQTSKLKALRLRCSGGM RLTATYRYILSCHCEECNS (SEQ ID NO. 6); P36A: MRKHVLAASFSMLSLLVIMGDTDSKTDSSFIMDSDARRCMRHHYVDSISHPLYKCSS KMVLLARCEGHCSQASRSEPLVSFSTVLKQPFRSSCHCCRPQTSKLKALRLRCSGGM RLTATYRYILSCHCEECNS (SEQ ID NO. 7), R37A: MRKHVLAASFSMLSLLVIMGDTDSKTDSSFIMDSDPARCMRHHYVDSISHPLYKCSS KMVLLARCEGHCSQASRSEPLVSFSTVLKQPFRSSCHCCRPQTSKLKALRLRCSGGM RLTATYRYILSCHCEECNS (SEQ ID NO. 8), R38A: MRKHVLAASFSMLSLLVIMGDTDSKTDSSFIMDSDPRACMRHHYVDSISHPLYKCSS KMVLLARCEGHCSQASRSEPLVSFSTVLKQPFRSSCHCCRPQTSKLKALRLRCSGGM RLTATYRYILSCHCEECNS (SEQ ID NO. 9); Y120A: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSGGM RLTATARYILSCHCEECNS (SEQ ID NO. 10), R121A: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSGGM RLTATYAYILSCHCEECNS (SEQ ID NO. 11), Y122A: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSGGM RLTATYRAILSCHCEECNS (SEQ ID NO. 12); or H127A: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSGGM RLTATYRYILSCACEECNS (SEQ ID NO. 13), E129A: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSGGM RLTATYRYILSCHCAECNS (SEQ ID NO. 14), E130A: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSCGGM RLTATYRYILSCHCEACNS (SEQ ID NO. 15); or combinations thereof. Any amino acid mutated in a multiple mutation is operable as a single mutation. Other sequence mutations operative herein are illustrated in FIGS. 5 and 6 of Smallwood, P M, et al., J Biol Chem, 2007: 282:4057-4068 or Ke, J et al. Genes& Dev. 2013: 27: 2305-2319. These mutations include K86E: MRKHVLAASFSMLSLLVIMGDTDSKTDSSFIMDSDPRRCMRHHYVDSISHPLYKCSS KMVLLARCEGHCSQASRSEPLVSFSTVLEQPFRSSCHCCRPQTSKLKALRLRCSGGM RLTATYRYILSCHCEECNS (SEQ ID NO. 16), R90E: MRKHVLAASFSMLSLLVIMGDTDSKTDSSFIMDSDPRRCMRHHYVDSISHPLYKCSS KMVLLARCEGHCSQASRSEPLVSFSTVLKQPFESSCHCCRPQTSKLKALRLRCSGGM RLTATYRYILSCHCEECNS (SEQ ID NO. 17), R97E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCEPQTSKLKALRLRCSGGM RLTATYRYILSCHCEECNS (SEQ ID NO. 18), K102E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSELKALRLRCSGGM RLTATYRYILSCHCEECNS (SEQ ID NO. 19), K104E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLEALRLRCSGGM RLTATYRYILSCHCEECNS (SEQ ID NO. 20), and R115E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSGGM ELTATYRYILSCHCEECNS (SEQ ID NO. 21). It is appreciated that other mutations at different amino acid sites are similarly operable. It is further appreciated that mutation of the conserved amino acid at any particular site is preferably mutated to glycine or alanine. It is further appreciated that mutation to any neutrally charged, charged, hydrophobic, hydrophilic, synthetic, non-natural, non-human, or other amino acid is similarly operable. The norrin of the present invention can be recombinant norrin, natural norrin, or synthetic norrin retaining binding properties to frizzled-4, LGR4, LGR5, LGR6, or combinations thereof. It will be recognized in the art that some amino acid sequences of the invention can be varied without significant effect of the structure or function of the protein. Thus, the invention further includes variations of the norrin which show substantial activity; such mutants include deletions, insertions, inversions, repeats, and type substitutions. A particularly well suited norrin mutant for the present invention is a truncate (SEQ ID NO. 2) with a mutation in at least one position 81-90 of SEQ ID NO: 1 that interferes with protease cleavage of the resulting protein. In some inventive embodiments, a truncate (SEQ ID NO. 2) that has one or more mutations at positions 84, 85, 86, 87, or 88 relative to SEQ ID NO: 1 affords a resulting norrin truncate-mutant that has a lower molecular weight than native norrin resulting in more rapid diffusion and a longer biological half-life owing to misfit as a substrate for one or more proteases that routinely degrade norrin in vivo. As a result, the norrin-truncate has a half-life that is more than 30% greater than native norrin, and in some embodiments between 50 and 500% greater than native norrin. Trypsin is known to cleavage deactivate native norrin.

Modifications and changes are optionally made in the structure (primary, secondary, or tertiary) of the Norrin protein which are encompassed within the inventive compound that may or may not result in a molecule having similar characteristics to the exemplary polypeptides disclosed herein. It is appreciated that changes in conserved amino acid bases are most likely to impact the activity of the resultant protein. However, it is further appreciated that changes in amino acids operable for receptor interaction, resistance or promotion of protein degradation, intracellular or extracellular trafficking, secretion, protein-protein interaction, post-translational modification such as glycosylation, phosphorylation, sulfation, and the like, may result in increased or decreased activity of an inventive compound while retaining some ability to alter or maintain a physiological activity. Certain amino acid substitutions for other amino acids in a sequence are known to occur without appreciable loss of activity.

In making such changes, the hydropathic index of amino acids are considered. According to the present invention, certain amino acids can be substituted for other amino acids having a similar hydropathic index and still result in a polypeptide with similar biological activity. Each amino acid is assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Without intending to be limited to a particular theory, it is believed that the relative hydropathic character of the amino acid determines the secondary structure of the resultant polypeptide, which in turn defines the interaction of the polypeptide with other molecules. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu).

The norrin and analogs can be further modified to contain additional chemical moieties not normally part of the protein. Those derivatized moieties can improve the solubility, the biological half-life, absorption of the protein, or binding affinity. The moieties can also reduce or eliminate any desirable side effects of the proteins and the like. An overview for those moieties can be found in Remington's Pharmaceutical Sciences, 20th ed., Mack Publishing Co., Easton, PA (2000).

The isolated norrin described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. (Zoeller et al., Proc. Nat'l. Acad. Sci. USA 81:5662-5066 (1984) and U.S. Pat. No. 4,588,585).

Norrin truncate of SEQ ID NO: 2 is observed to be effective in increasing cellular junction levels of claudin-5 and VE-cadherins at concentrations of 10 to 1000 ng/ml.

The present invention is also directed to pharmaceutical compositions comprising an effective amount of norrin alone or in combination with a pharmaceutically acceptable carrier, excipient or additive. Particularly favored derivatives are those that increase the bioavailability of norrin administered to a mammal (e.g., by allowing ocularly and aurally of choroidal administered norrin to be more readily absorbed into the blood) or which enhance delivery of the norrin to a biological compartment (e.g., the retina, the ear) relative to the native protein.

To prepare the pharmaceutical compositions according to the present invention, a therapeutically effective amount of norrin is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, aural, topical or parenteral, including gels, creams ointments, lotions and time released implantable preparations, among numerous others.

Solutions or suspensions used for ocular, aural, parenteral, intradermal, subcutaneous, or topical application can include the following components: a sterile diluent such as water for injection, saline solution, fixed oils, polyethylene glycols, glycerine, propylene glycol or other synthetic solvents: antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, poly anhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

Formulations suitable for topical administration to the skin may be presented as ointments, creams, gels and pastes including the ingredient to be administered in a pharmaceutical acceptable carrier. A preferred topical delivery system is a transdermal patch containing the ingredient to be administered.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier will usually comprise sterile water or aqueous sodium chloride solution, though other ingredients including those which aid dispersion may be included. Of course, where sterile water is to be used and maintained as sterile, the norrin and carriers must also be sterilized. Injectable suspensions may also be prepared, in which case appropriate liquid carriers, suspending agents and the like may be employed.

Formulations suitable for parenteral or ocular administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophiized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include topical, ocular, aural, parenteral, intramuscular, intravenous, sub-cutaneous, intrachoroidal, or transdermal (which may include a penetration enhancement agent).

Application of the subject therapeutics may be local, so as to be administered at the site of interest. Various techniques can be used for providing the subject norrin at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject norrin, the subject norrin may be painted onto the organ, or may be applied in any convenient way.

Norrin may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment. More specifically, the norrin is administered through an ocular device suitable for direct implantation into the vitreous of the eye or through an aural device suitable for direct implantation into the inner ear. Such devices of the present invention are surprisingly found to provide sustained controlled release of various norrin to treat the eye and ear without risk of detrimental local and systemic side effects. An object of the present ocular and aural method of delivery is to maximize the amount of drug contained in an intraocular or intra-aural device while minimizing its size in order to prolong the duration of the implant. See, e.g., U.S. Pat. Nos. 5,378,475; 5,773,019; 6,001,386; 6,217,895, 6,375,972, and 6,756,058.

Other methods of delivery of norrin include: an ocular delivery system that could be applied to an intra-ocular lens to prevent inflammation or posterior capsular opacification, an ocular delivery system that could be inserted directly into retinal ganglion cells, the vitreous, under the retina, or onto the sclera, and wherein inserting can be achieved by injecting the system or surgically implanting the system, an aural delivery system that could be applied on or inserted directly into various structures of the inner ear illustratively including, the cochlea, the organ of Corti, the stria vascularis, spiral ganglion cells, or the auditory nerve, and wherein inserting can be achieved by injecting the system or surgically implanting the system, a sustained release drug delivery system, and a method for providing controlled and sustained administration of an agent effective in obtaining a desired local or systemic physiological or pharmacological effect comprising surgically implanting a sustained release drug delivery system at a desired location.

Examples include, but are not limited to the following: a sustained release drug delivery system comprising an inner reservoir containing norrin, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end. A method for administering norrin to a segment of an eye, includes implanting a sustained release device to deliver norrin to the vitreous of the eye or choroid, or an implantable, sustained release device for administering a compound of the invention to a segment of an eye or choroid; a sustained release drug delivery device includes a) a drug core containing norrin; b) at least one unitary cup essentially impermeable to the passage of the agent that surrounds and defines an internal compartment to accept the drug core, the unitary cup including an open top end with at least one recessed groove around at least some portion of the open top end of the unitary cup; c) a permeable plug which is permeable to the passage of norrin, the permeable plug is positioned at the open top end of the unitary cup wherein the groove interacts with the permeable plug holding it in position and closing the open top end, the permeable plug allowing passage of the agent out of the drug core, through the permeable plug, and out the open top end of the unitary cup. A method for administering norrin to a segment of an ear, includes implanting a sustained release device to deliver norrin to the inner ear, or an implantable, sustained release device for administering a compound of the invention to a segment of an ear; a sustained release drug delivery device includes a) a drug core containing norrin; b) at least one unitary cup essentially impermeable to the passage of the agent that surrounds and defines an internal compartment to accept the drug core, the unitary cup including an open top end with at least one recessed groove around at least some portion of the open top end of the unitary cup; c) a permeable plug which is permeable to the passage of norrin, the permeable plug is positioned at the open top end of the unitary cup wherein the groove interacts with the permeable plug holding it in position and closing the open top end, the permeable plug allowing passage of the agent out of the drug core, through the permeable plug, and out the open top end of the unitary cup. A sustained release norrin delivery device includes an inner core norrin having a desired solubility and a polymer coating layer, the polymer layer being permeable to norrin, wherein the polymer coating layer completely covers the inner core.

Norrin may be administered as microspheres. For example, norrin may be purchased from R&D Systems, Minneapolis, Minn., or cloned, expressed and purified is loaded into biodegradable microspheres substantially as described by Jiang, C, et al., Mol. Vis., 2007; 13:1783-92 using the spontaneous emulsification technique of Fu, K, et al., J. Pharm. Sci., 2003; 92:1582-91. Microspheres are synthesized and loaded by dissolving 200 mg of 50:50 poly(lactide-co-glycolic acid) (PLGA) in 5 ml of 4:1 volume ratio trifluoroethanol:dichloromethane supplemented with 8 mg magnesium hydroxide to minimize protein aggregation during encapsulation. 10 μg norrin may be reconstituted in 300 μl 7 mg bovine serum albumin (BSA) and 100 mg docusate sodium (Sigma-Aldrich. St. Louis, Mo.) dissolved in 3 ml PBS. The solution may be vortexed and poured into 200 ml of 1% (w/v) polyvinyl alcohol (PVA, 88% hydrolyzed) with gentle stirring. Microspheres may be hardened by stirring for three hours, collected by centrifugation, and washed three times to remove residual PVA. If the microspheres are not to be immediately injected they are rapidly frozen in liquid nitrogen, lyophilized for 72 h, and stored in a dessicator at −20° C. Norrin containing microspheres exhibit average diameters of 8μιη as determined by a particle size. Norrin may also be administered by intravitreal injection. For example, norrin in solution, may be packaged into microspheres as described above, or expressed in cells, or in purified form in solution may be exposed to the retina or ear by intravitreal or inner-ear injection substantially as described by Jiang, 2007. Intravitreal or inner ear injection may be performed under general anesthesia using an ophthalmic or otolaryngolic operating microscope (Moller-Wedel GmbH, Wedel, Germany) using beveled glass micro-needles with an outer diameter of approximately 100 μm. Microsphere suspensions are prepared in PBS at 2 and 10% (w/v) and briefly vortexed immediately before injection to ensure a uniform dispersion. For intravitreal injection, a 30-gauge hypodermic needle may be used to perforate the sclera 1.5 mm behind the limbus. Five microliters of test sample is optionally injected by way of this passage into the vitreous using a 50 μl Hamilton Syringe (Hamilton Co, Reno, Nev.). To ensure adequate delivery and prevent shock the needle is held in place for one min after the injection is completed and subsequently withdrawn slowly. In addition, paracentesis may be simultaneously performed to relieve pressure and thereby prevent reflux.

Norrin may also be administered by delivery to the retina or ear by a controlled release delivery system. An implantable controlled release delivery system is described in U.S. Patent Application Publication 2005/0281861 and is packaged into such as system at 100 μg per final formulated capsule. For example, for delivery to the retina, a norrin containing drug delivery systems may be placed in the eye using forceps or a trocar after making a 2-3 mm incision in the sclera. Alternatively, no incision may be made and the system placed in an eye by inserting a trocar or other delivery device directly through the eye. The removal of the device after the placement of the system in the eye can result in a self-sealing opening. One example of a device that is used to insert the implants into an eye is disclosed in U.S. Patent U.S. Pat. No. 6,899,717B1 which is incorporated herein by reference. The location of the system may influence the concentration gradients of therapeutic component or drug surrounding the element, and thus influence the release rates (e.g., an element placed closer to the edge of the vitreous may result in a slower release rate). Thus, it is preferred if the system is placed near the retinal surface or in the posterior portion of the vitreous.

Preferred unit dosage formulations are those containing a daily dose or unit, daily sub-dose, as hereinabove recited, or an appropriate fraction thereof, of the administered ingredient.

The dosage regimen for norrin invention is based on a variety of factors, including the degree of BRB leakage, the route of administration, ocular volume, aural volume, macular separation volume, and the particular norrin employed. Thus, the dosage regimen may vary widely, but can be determined routinely using standard methods.

In certain embodiments, norrin is administered once daily; in other embodiments, norrin is administered twice daily; in yet other embodiments, norrin is administered once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

Pharmaceutically acceptable carriers, excipients, or diluents illustratively include saline, buffered saline, dextrose, water, glycerol, ethanol, sterile isotonic aqueous buffer, or combinations thereof.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as PLGA, polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

Examples of preservatives include, but are not limited to, parabens, such as methyl or propyl p-hydroxybenzoate and benzalkonium chloride.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Any of the above-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours. 6 hours, 8 hours, 10 hours, 12 hours, 16 hours. 24 hours, 48 hours, 72 hours, or more after administration of the pharmaceutical compositions to the subject.

When norrin is administered as a pharmaceutical to humans or animals, norrin can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient.

Exemplary ocular and aural dose ranges include 0.1 ng (0.0000001 mg) to 250 mg per day, 0.0001 mg to 100 mg per day, 1 mg to 100 mg per day. 10 mg to 100 mg per day, 1 mg to 10 mg per day, and 0.01 mg to 10 mg per day. A preferred dose of an agent is the maximum that a patient can tolerate and not develop serious or unacceptable side effects. In certain inventive embodiments, the therapeutically effective dosage produces an ocular or aural concentration of norrin of from about 0.1 ng/ml to 100 μg/ml. In certain inventive embodiments, 50 nM to 1 μM of an agent is administered to a subject eye or ear. In related embodiments, about 50-100 nM, 50-250 nM, 100-500 nM, 250-500 nM, 250-750 nM, 500-750 nM, 500 nM to 1 μM, or 750 nM to 1 μM of an norrin is administered to a subject eye or ear.

Determination of an effective amount is well within the capability of those skilled in the art, especially in light of the detailed disclosure provided herein. Generally, an efficacious or effective amount of a norrin is determined by first administering a low dose of the agent(s) and then incrementally increasing the administered dose or dosages until a desired effect (e.g., nerve regeneration in the ear and/or retina) is observed in the treated subject, with minimal or acceptable toxic side effects. Applicable methods for determining an appropriate dose and dosing schedule for administration of a pharmaceutical composition of the present invention are described, for example, in Goodman and Oilman's The Pharmacological Basis of Therapeutics, Goodman et al., eds., 11th Edition, McGraw-Hill 2005, and Remington: The Science and Practice of Pharmacy. 20th and 21st Editions, Gennaro and University of the Sciences in Philadelphia, Eds., Lippencott Williams & Wilkins (2003 and 2005), each of which is hereby incorporated by reference.

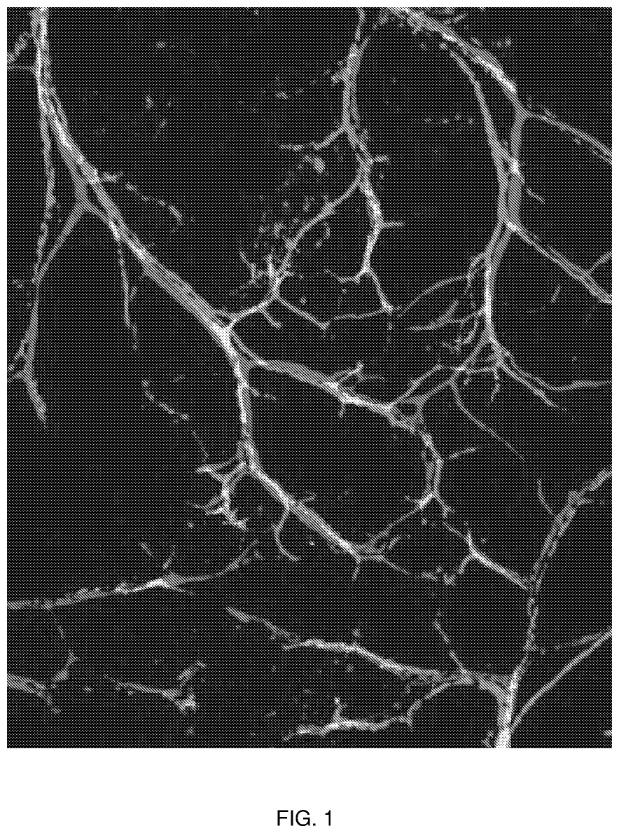

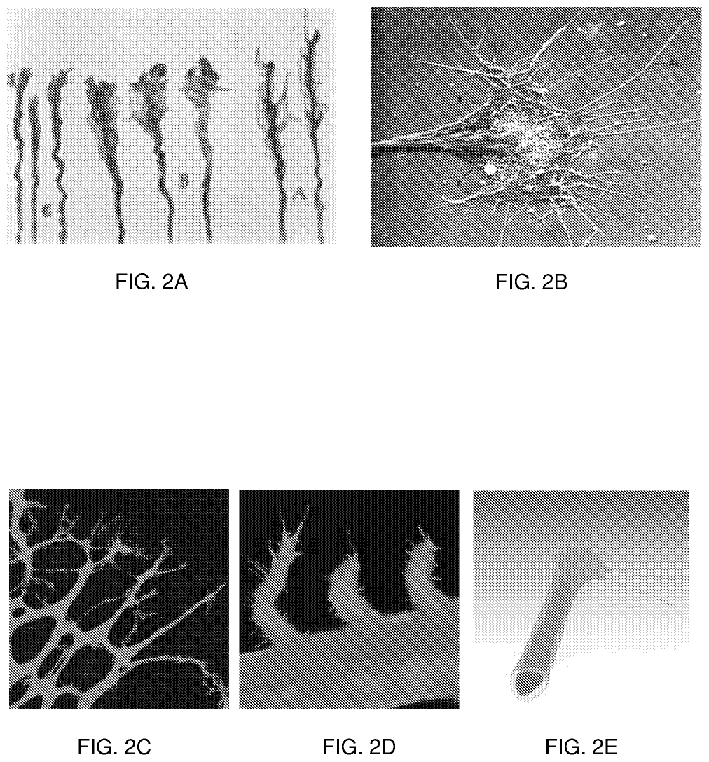

Referring now to the figures, FIG. 1 illustrates how angiogenesis and neurogenesis occur simultaneously. In FIG. 1, vascular structure is shown in red and nerves are shown in green, and are readily seen tracking towards a destination. FIGS. 2A-2E illustrate stereotyped axon and vessel navigation where overlapping guidance cues direct growth of both the nerves and blood vessels.

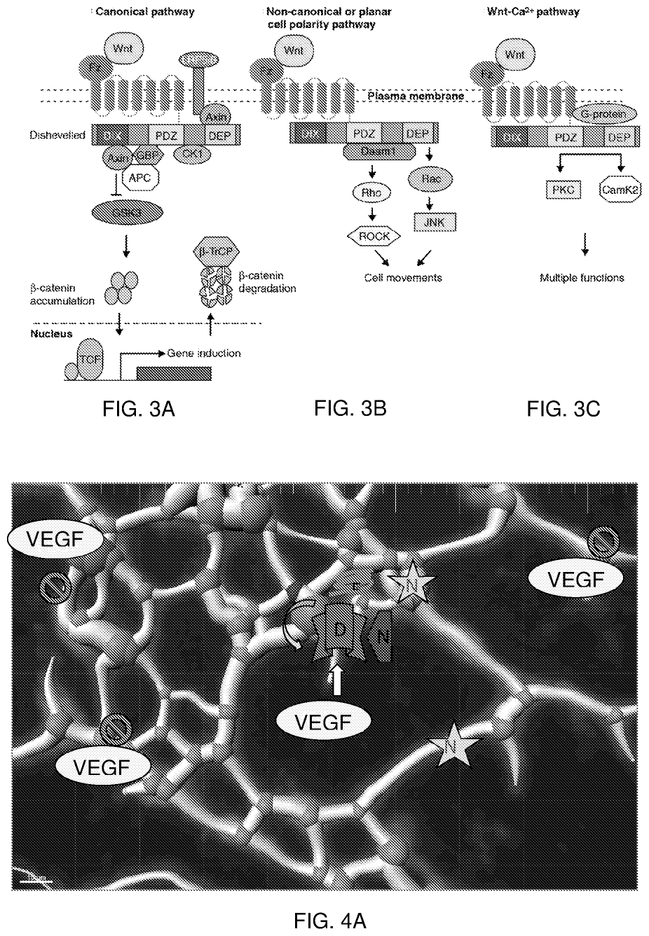

As previously described, norrin (Pro-micronorrin) activates Wnt pathways, where with respect to blood vessels the norrin binds the Fzd4 receptor on the retinal endothelial cells, and in neurons the norrin binds the LGR4 receptor on retinal ganglion cells and aural nerve cells illustratively including spiral ganglion cells and the auditory nerve. Without intending to be bound by a particular theory, FIGS. 3A-3C illustrate by way of schematics the process of norrin angiogenesis through the Fzd4 receptor on endothelial cells, where FIG. 3A illustrates a canonical pathway, FIG. 3B is a non-canonical or planar cell polarity pathway, and FIG. 3C is a Wnt-Ca²⁺ pathway.²⁹ Abbreviated terms for various proteins and receptors have the conventional meaning. These include LRP5/6 (LDL Receptor Related Protein 5 and or 6), DIX domain (Dishevelled and Axin), PDZ (Post-synaptic density protein-95), DEP (Dishevelled, Egl-10 and Pleckstrin), GBP (guanylate-binding protein), CK1 (Casein kinase 1), APC protein (adenomatous polyposis coli protein), GSK3 (glycogen synthase kinase-3), β-TrCP (beta-transducin repeats-containing proteins), TCF (T-cell factor/lymphoid enhancer factor), DAAM1 (disheveled-associated activator of morphogenesis 1), Rho (GTPases that acts as molecular switches that cycle between an active (GTP-bound) and an inactive (GDP-bound) conformation under the control of guanine nucleotide exchange factors (GEFs) and GTPase-activating proteins (GAPs)), ROCK (rho-associated protein kinase), Rac (Ras-related C3 botulinum toxin substrate—GAP), JNK (Jun N-terminal kinase), G-protein (guanine nucleotide-binding protein), PKC (protein kinase C), and CamK2 (Ca2+/calmodulin-dependent protein kinase II).

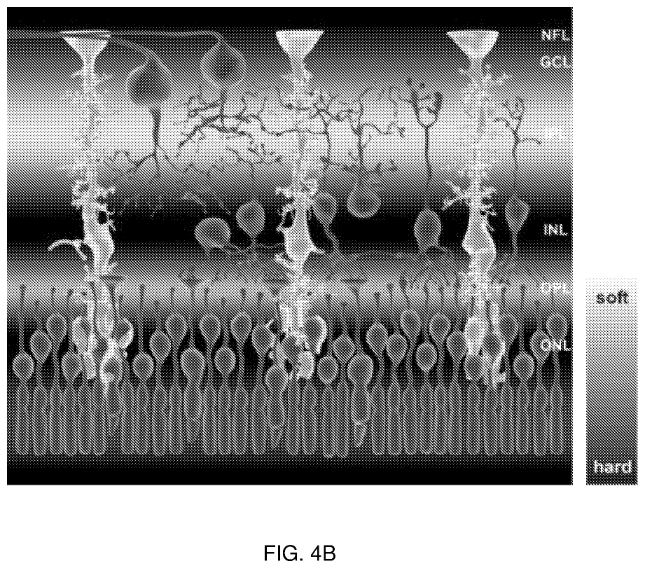

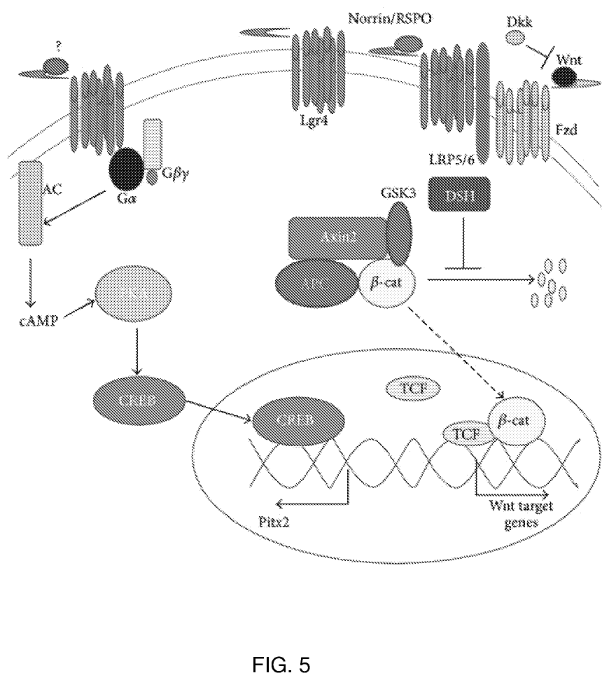

FIG. 4A illustrates norrin-mediated growth of endothelial buds. In modulated angiogenesis the following occurs: endothelial buds (Eb) require vascular endothelial growth factor (VEGF) to grow, norrin binds Fzd4 receptor on RECs, activates expression of DLL4 receptor on RECs, DLL4 binds Notch1 which sensitizes Eb to VEGF, and simultaneously desensitizes surrounding Ebs to VEGF. Norrin promotes glial cell growth which is needed for angiogenesis and neurogenesis. Glial cells are a shared pathway for both angiogenesis and neurogenesis. FIG. 4B depicts endothetial cell differentiation into phalanx, stalk and tip cells in response to norrin administration to regenerate retinal neurons where the respective layers are NFL (nerve fiber layer) GCL (ganglion cell layer), IPL (inner plexiform layer), INL (inner nuclear layer), OPL (outer plexiform layer), and ONL (outer nuclear layer) with vascular structure shown in red and nerves shown in green. The grayscale legend denotes a relative hardness of a given layer.

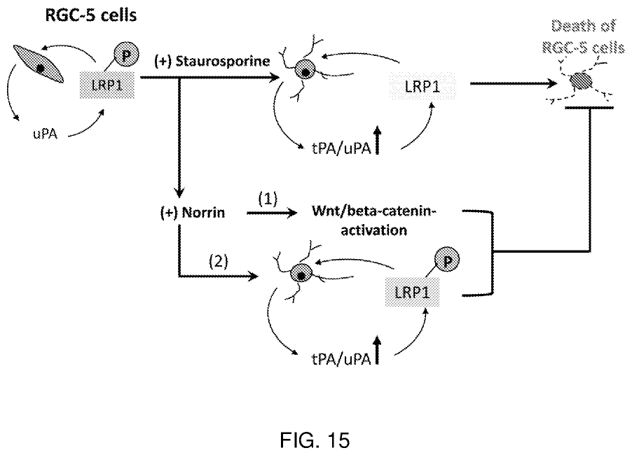

While not intending to be bound to a particular mechanism, FIG. 5 illustrates norrin mediated FZD4, LGR4, LGR5, and LGR6 activation on retinal ganglion cells.³⁰ Norrin stimulates differentiation and survival of resident stem cells, where, as illustrated in FIG. 15, chx10/Pax6 co-immunostain is specific for retinal progenitor cells and observed with norrin treatment following an injury. The AA change is Lys86Pro, which eliminates an internal protease cleavage site in the large loop section of norrin. Norrin is denoted as a red horizontal oval. Abbreviations in FIG. 5 include those detailed with respect to FIGS. 3A-3C, as well as RSPO (R-spondin protein), Dkk (Dickkopf-related protein), Lgr4 (leucine-rich repeat-containing G-protein coupled receptor 4), AC (adenylate cyclase), cAMP (cyclic adenosine monophosphate), PKA (protein kinase A), CREB (cAMP-responsive element binding protein), beta-cat beta catenin), DSH (disheveled protein), and Pitx2 (bicoid-related transcription factor).

FIG. 6 is a bar graph illustrating norrin gene expression in embroids. At day 21 it can be seen that cells begin differentiation into neurons. Norrin mRNA expression also significantly increases. This evidence indicates that norrin plays a role in neurogenesis and that norrin can be used to stimulate nerve regeneration in the ear and the retina in order to treat, mitigate, prevent, and reverse the effects of nerve damage and nerve regeneration in the ear and retina illustratively including hearing and vision loss.

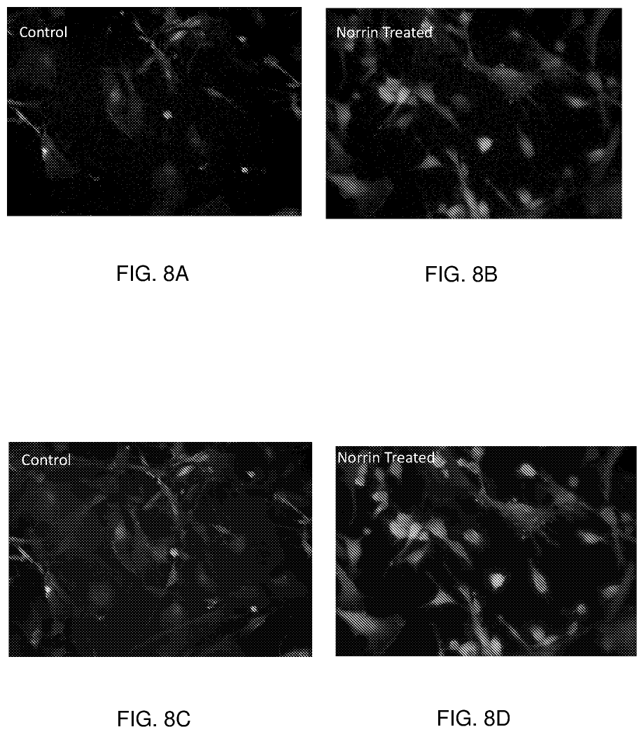

FIGS. 7A-7D are a series of four photographs each with two control panels (FIGS. 7A, 7C) and two norrin truncate-mutant (SEQ ID NO. 2 with position 84 mutation) treated panels (FIGS. 7B, 7D) illustrating neurofilament-L (NFL) immunostain (green) of differentiated embroid bodies (2 weeks). As can be seen, the two norrin treated panels display a significant increase in protein production demonstrating neuronal growth.

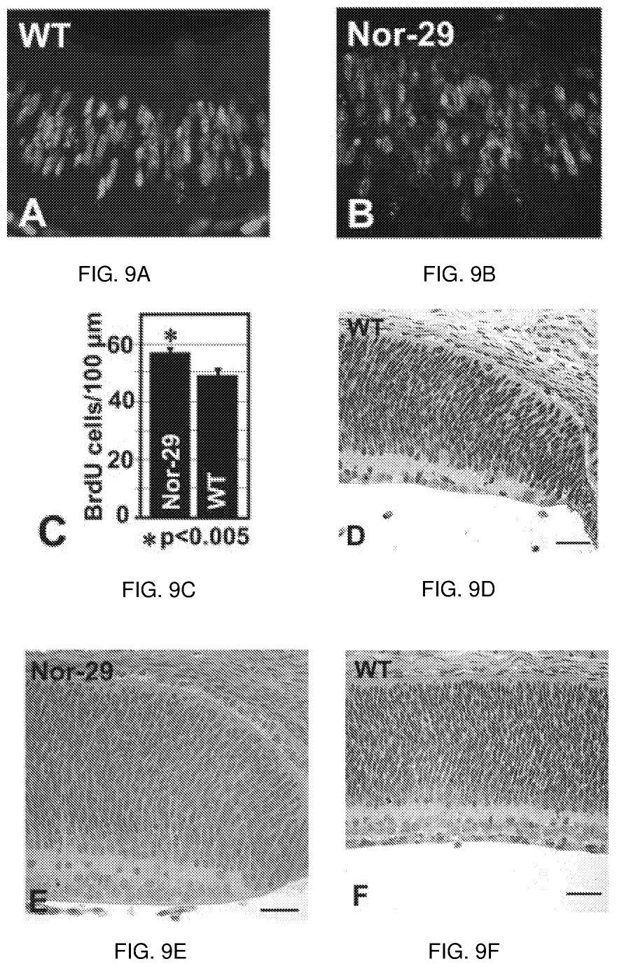

FIGS. 8A-8D are a series of four photographs each with two control panels (8A, 8C) and two norrin truncate-mutant treated panels (8B, 8D) as used in FIG. 7, illustrating nestin immunostain (green) of differentiated embroid bodies (2 weeks). As can be seen, the two norrin truncate-mutant treated panels display a significant increase in protein production.

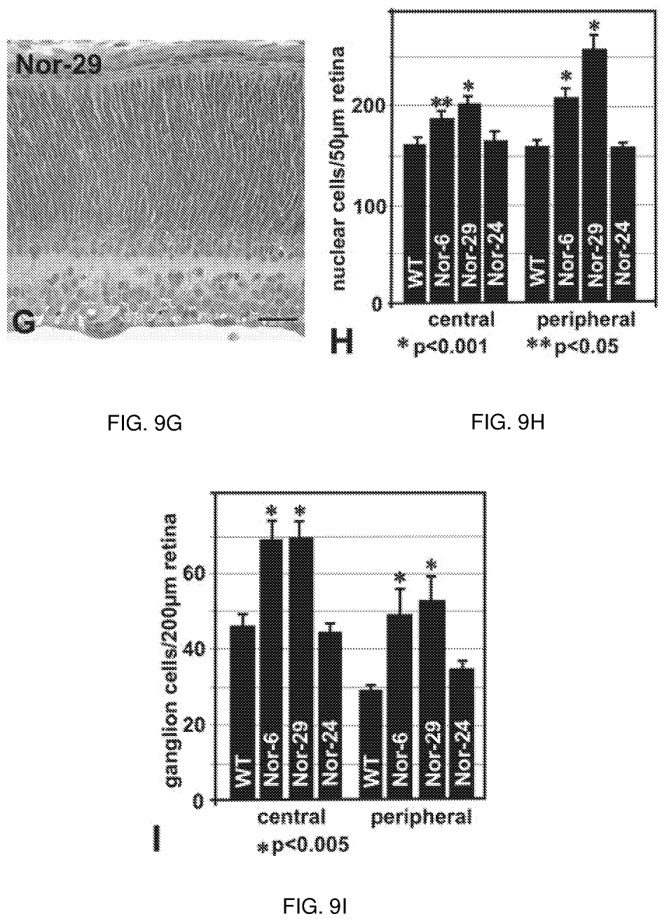

FIGS. 9A-9I are a series of six photographs (9A, 9B, 9D, 9E, 9F, 9G) and three graphs (9C, 9H, 9I) illustrating an increase in proliferation of retinal progenitor cells and retinal thickness in mice with ectopic expression of norrin. As can be seen in FIG. 9I, central retinal ganglion cells have significantly increased with the expression of norrin. This evidence further indicates that norrin treatment can stimulate nerve regeneration in organs of the retina and the ear.

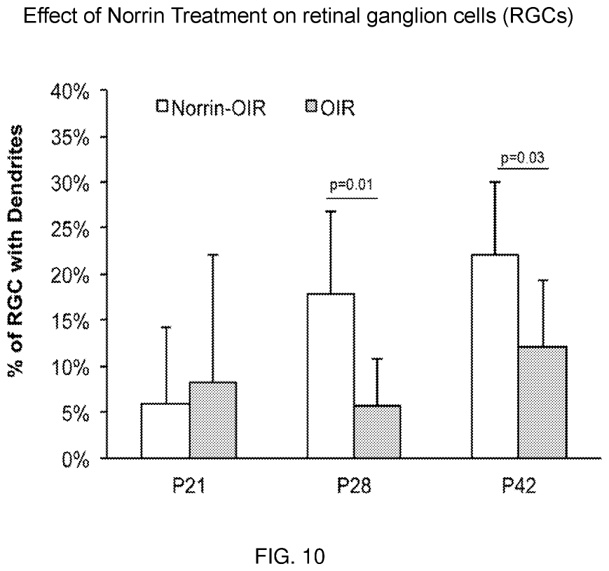

FIG. 10 is a graph illustrating the effect of norrin treatment on retinal ganglion cells (RGCs). A single intravitreal injection of norrin was given at p14. As can be seen from the data, norrin significantly increases the presence of mature RGCs with dendrites. Furthermore, this effect was sustained without the need for an additional intravitreal injection of norrin after p14.

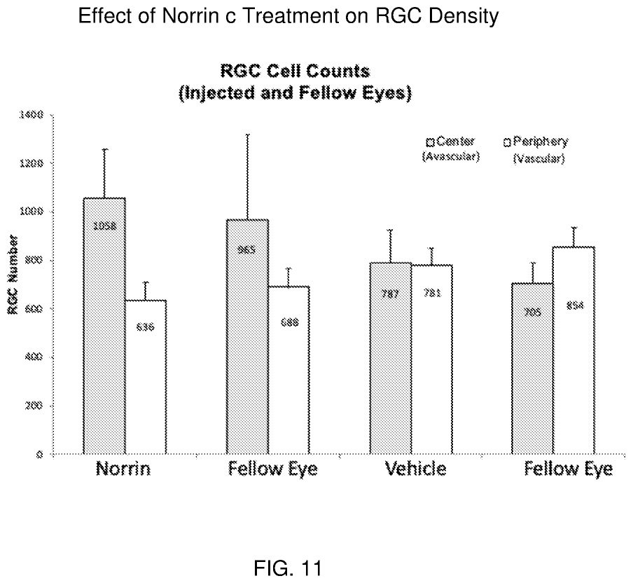

FIG. 11 is a graph illustrating the effect of norrin truncate-mutant treatment on RGC density after a single injection of norrin at p14. It was observed that the avascular retina (center) shows the greatest benefit in increase in RGC density after a single injection of norrin truncate-mutant at p14. Similar to other intravitreal therapies, there is a fellow eye effect with norrin treatment.

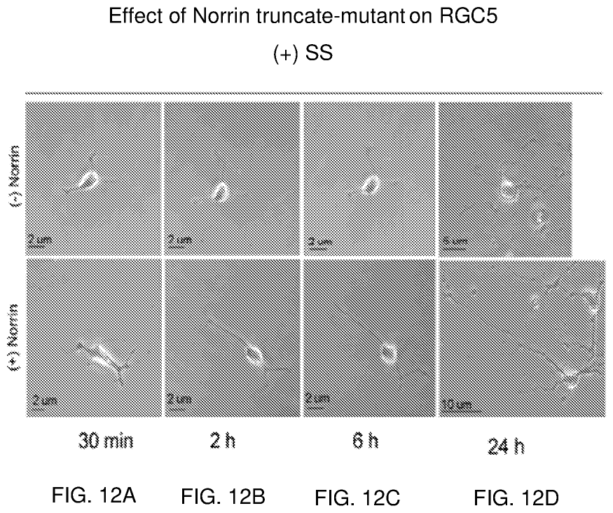

FIGS. 12-14 illustrate the effect of norrin truncate-mutant on RGC5. FIGS. 12A-12D illustrate that norrin truncate-mutant promotes cell survival and also promotes dendrite and axon growth as more dendrites and axons are observed at 2, 6, and 24 hours relative to 30 minutes in the norrin truncate-mutant treated cells compared to the cells that were not norrin truncate-mutant treated. Increased intercellular communication was also observed.

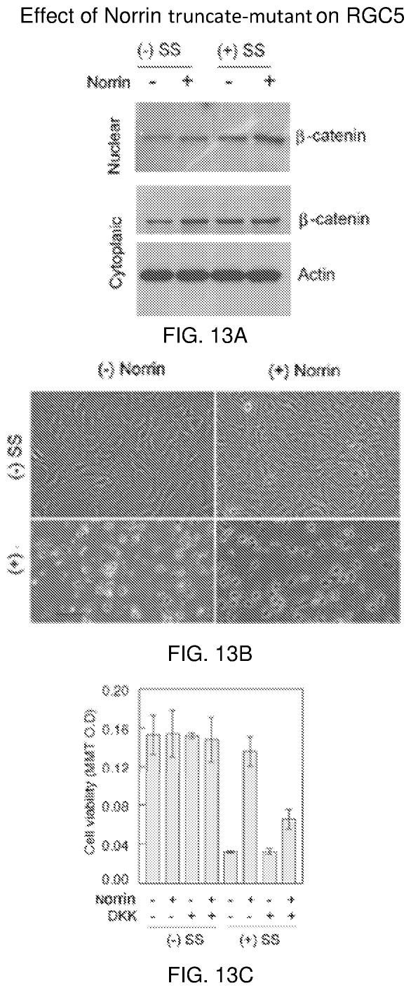

FIGS. 13A-C illustrate that norrin truncate-mutant increases nuclear β-catenin (FIG. 13A) and promotes cell survival (FIG. 13C), whereas Wnt-inhibition (DKK) decreases cell survival as DKK partially blocks norrin's cell protection as also shown in FIG. 13C. Some RGC-5 cells are treated with 2.0 μM staurosporine (+)SS, a broad-spectrum protein kinase-C inhibitor, to induce growth arrest, differentiation, and elevated levels of tissue plasminogen activator (tPA) and urokinase plasminogen activator (uPA), and compared to other cells absent such a treatment (−)SS. This evidence supports native norrin's role in activating Wnt-signaling and stimulating nerve regeneration, as shown in the micrographs of FIG. 13B. Which of the three Wnt intracellular pathways transmits the signal may depend on cellular context. The presence of various receptor complex components may determine which pathway is activated. For example, LRP5 is a requirement for canonical signaling in general and TSPAN12 enhances canonical activation by norrin.¹⁴ It appears that norrin has a greater affinity for the Fzd4 receptor alone, and the effective removal of LRP5 by DKK1 increases norrin's binding to the canonical receptor complex. In the combined (norrin+DKK1) injection, it may be envisioned that norrin binds to a receptor complex that cannot be activated, given that the coreceptor (LRP5) has been bound by DKK1. Therefore, DKK1 binding of LRP5 may result in increased affinity of norrin for the canonical receptor complex, effectively sequestering norrin away from the noncanonical pathways. This would result in decreased noncanonical signaling and defective canonical signaling, essentially canceling any effect of norrin. In this scenario the rescue effect seen with DKK1 alone may be masked by the norrin binding. It appears that norrin may competitively inhibit binding of the endogenous Wnts to the Fzd4 receptor.

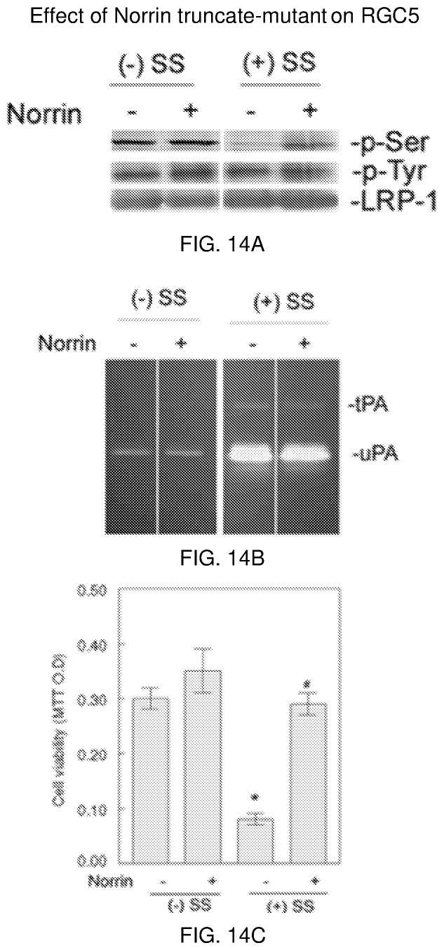

FIGS. 14A-14C illustrate the effect of norrin on RGC5, particularly, that cell viability after norrin treatment increases relative to cells not treated with norrin truncatate-mutant as evidenced by protein expression levels in FIG. 14A, tPA and uPA levels in FIG. 14B, and the associated cell viability as shown in FIG. 14C. This evidence supports norrin's role in preventing, mitigating, and possibly reversing the effects of nerve degeneration and damage.

FIG. 15 illustrates a proposed mechanisms by which norrin promotes neuron protection through LRP1. Norrin stimulates Wnt/β-catenin signaling which in-turn promotes neuron protection through LRP1.

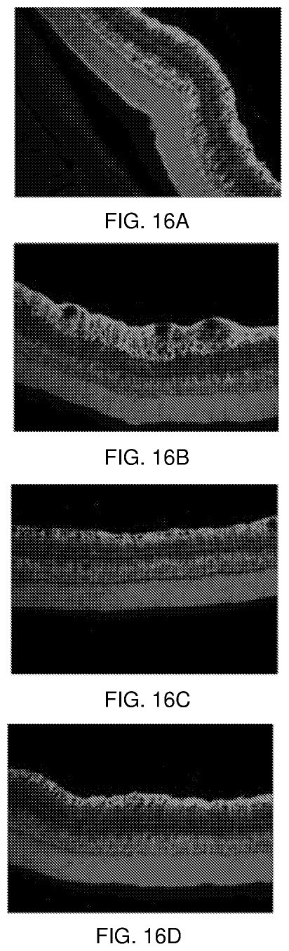

FIGS. 16A-16C are a series of four photographs illustrating a control panel (FIG. 16A), an NMDA (N-methyl-D-aspartate) panel (FIG. 16B), an NMDA-Wnt3 panel (FIG. 16C), and an NMDA-Norrin panel (FIG. 16D). The red immunostain is glutamine synthetase (a Miller cell marker), and the blue immunostain is a nuclear stain. This evidence illustrates the positive effect of norrin treatment following NMDA injury. NMDA is known to induce retinal cell death and RGC degeneration-effects that are extendible to aural ganglion and associated hearing loss.

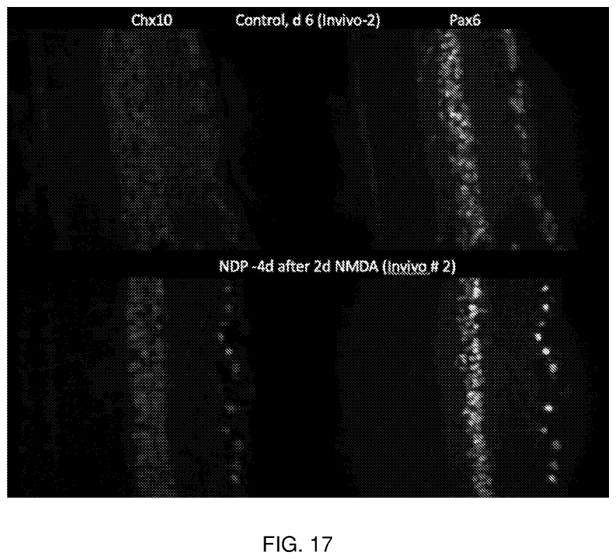

FIG. 17 is a photograph illustrating a co-immunostain of Chx10 and Pax6 which is a co-immunostain specific for retinal progenitor cells. This co-immunostain is observed with norrin truncation-mutant treatment following NMDA injury and is evidence that norrin stimulates cellular differentiation and survival of resident stem cells after injury. Without intending to be bound by a particular theory, it is believed that norrin's stimulation of cellular differentiation and survival of resident stem cells after injury plays a role in norrin's ability to stimulate nerve regeneration in damaged nerves and nerves that have degenerated via the chemical pathways discussed hereinabove.

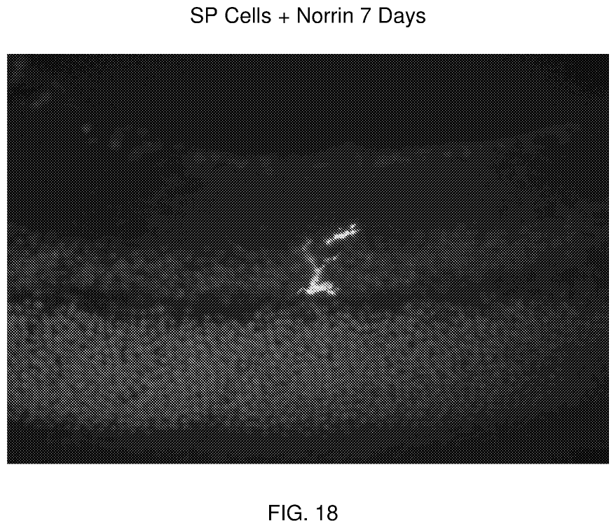

FIG. 18 is a photograph illustrating side population of hematopoetic stem cells—GFP cells introduced with intravitreal injection followed by intravitreal injection of norrin 7 days after. Without intending to be bound by a particular theory, it is believed that norrin acts as a neuroprotective for retinal and aural nerve survival upon injury which involves stimulation of Wnt/β-catenin signaling, which activates production of Müller glia (in the retina) and increase synthesis of neuroprotective growth factors in damaged nerves in the retina and the ear.

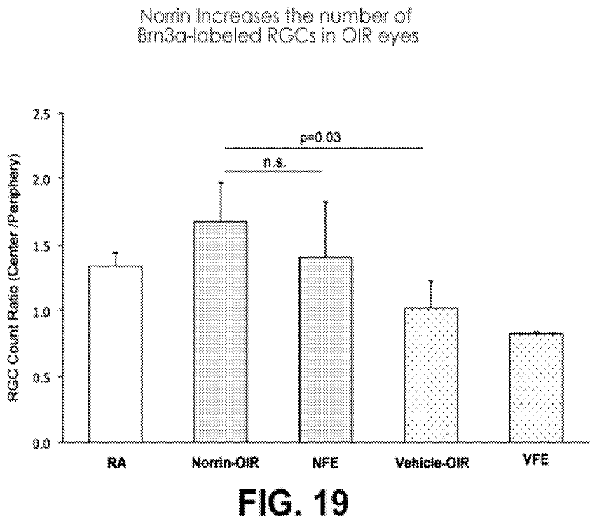

FIG. 19 is a graph illustrating that norrin increases the number of Brn3a-labeled RGCs in OIR eyes. The ratio (center/periphery) of the RGC cell density was significantly higher in norrin-injected OIR eyes (Norrin) compared to vehicle-injected OIR eyes (Vehicle) (p=0.03). Other groups shown are: room-air (RA), fellow non-injected eye of norrin-treated mice (NFE), fellow non-injected eye of vehicle-treated mice (VFE).

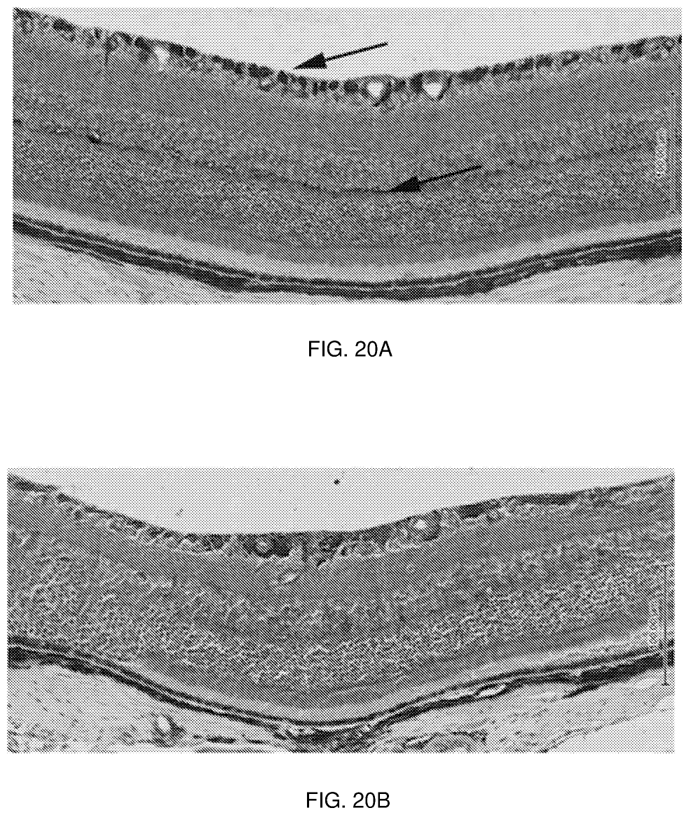

FIGS. 20A and 20B are a series of two photographs illustrating LGR4 immnostained in the RGCs, the inner edge of the amacrine cells, and the outer plexiform layer of the retina. As discussed hereinabove, evidence shows that norrin binds to LGR4, LGR5, LGR6 receptors, and specifically activates the LGR4 receptor which stimulates Wnt signaling and neurogenesis.

The highly localized expression of norrin in the retina, cochlea, and central nervous system during development suggests a highly specific role for norrin in the appropriate maturation of these particular tissues. There are other nonspecific Wnt ligands that are able to bind both the Fzd4 and LRP5 (receptor and coreceptor for norrin), but these have been shown to be upregulated during pathologic angiogenesis.³¹ Clinical studies and animal models clearly show that lack of norrin expression in the eye results in severe abrogation of retinal development, indicating that Wnt pathway activation alone (by other Wnt ligands) is not sufficient for normal retinal development and vasculaturization.³²⁻³⁵ Mice are recognized as an animal model for retinal and aural nerve damage in humans.^{36,37}

Based on the experimental findings presented herein, norrin may represent a unique molecule that is able to function as both a Wnt-ligand and a growth factor and regulate angiogenesis and neurogenesis in a fashion that mimics that seen in the developing eye and ear. Norrin may also stimulate regeneration of inner ear sensory hair cells and support their integration into nerve cells. This has significant implication in the treatment of many eye and ear diseases characterized by anomalous vasculature and anomalous nerve development. These conditions are seen in the inherited vitreoretinopathies, illustratively including FEVR, Norrie disease, and persistent fetal vasculature, as well as retinopathy of prematurity, diabetic retinopathy, retinal artery and vein occlusions, inner ear hair loss, and cochlear abnormalities.

As a person skilled in the art will recognize from the previous detailed description and from the figures and claims, modifications and changes can be made to the preferred embodiments of the invention without departing from the scope of this invention defined in the following claims.

### REFERENCES

1. Schuback D E., Chen Z Y., Craig I W., Breakeeld X O., Sims K B.; Mutations in the Norrie disease gene. Hum Mutat. 1995; 5: 285-292.
2. Meindl A., Berger W., Meitinger T.; Norrie disease is caused by mutations in an extracellular protein resembling C-terminal globular domain of mucins. Nat Genet. 1992; 2: 139-143.
3. Berger W., van de Pol D., Warburg M.; Mutations in the candidate gene for Norrie disease. Hum Mol Genet. 1992; 1: 461-465.
4. Shastry B S., Hejtmancik J F., Plager D A., Hartzer M K., Trese M T.; Linkage and candidate gene analysis of X-linked familial exudative vitreoretinopathy. Genomics. 1995; 27: 341-344.
5. Chen Z Y., Battinelli E M., Fielder A.; A mutation in the Norrie disease gene (NDP) associated with X-linked familial exudative vitreoretinopathy. Nat Genet. 1993; 5: 180-183.
6. Black G C., Perveen R., Bonshek R.; Coats' disease of the retina (unilateral retinal telangiectasis) caused by somatic mutation in the NDP gene: a role for norrin in retinal angiogenesis. Hum Mol Genet. 1999; 8: 2031-2035.
7. Talks S J., Ebenezer N., Hykin P.; De novo mutations in the 5′ regulatory region of the Norrie disease gene in retinopathy of prematurity. J Med Genet. 2001; 38: E46.
8. Hutcheson K A., Paluru P C., Bernstein S L.; Norrie disease gene sequence variants in an ethnically diverse population with retinopathy of prematurity. Mol Vis. 2005; 11: 501-508.
9. Hiraoka M., Berinstein D M., Trese M T., Shastry B S.; Insertion and deletion mutations in the dinucleotide repeat region of the Norrie disease gene in patients with advanced retinopathy of prematurity. J Hum Genet. 2001; 46: 178-181.
10. Xu Q., Wang Y., Dabdoub A.; Vascular development in the retina and inner ear: control by Norrin and Frizzled-4, a high-anity ligand-receptor pair. Cell. 2004; 116: 883-895.
11. Descamps B., Sewduth R., Ferreira Tojais N.; Frizzled 4 regulates arterial network organization through noncanonical Wnt/planar cell polarity signaling. Circ Res. 2012; 110: 47-58.
12. Yao R., Natsume Y., Noda T.; MAGI-3 is involved in the regulation of the JNK signaling pathway as a scaffold protein for frizzled and Ltap. Oncogene. 2004; 23: 6023-6030.
13. Robitaille J., MacDonald M L., Kaykas A.; Mutant frizzled-4 disrupts retinal angiogenesis in familial exudative vitreoretinopathy. Nat Genet. 2002; 32: 326-330.
14. Junge H., Yang S., Burton J.; TSPAN12 regulates retinal vascular development by promoting norrin, but not Wnt-induced FZD4/β-catenin signaling. Cell. 2009; 139: 299-311.
15. Berger W.; Molecular dissection of Norrie disease. Acta Anat (Basel). 1998; 162: 95-100.
16. Black G., Redmond R M.; The molecular biology of Norrie's disease. Eye. 1994; 8: 491-496.
17. McDonald N Q., Hendrickson W A.; A structural superfamily of growth factors containing a cystine knot motif. Cell. 1993; 73: 421-424.
18. Ohlmann A., Seitz R., Braunger B., Seitz D., Bosl M R., Tamm E R.; Norrin promotes vascular regrowth after oxygeninduced retinal vessel loss and suppresses retinopathy in mice. J Neurosci. 2010; 30: 183-193.
19. Smith L E., Wesolowski E., McLellan A.; Oxygen-induced retinopathy in the mouse. Invest Ophthalmol Vis Sci. 1994; 35: 101-111.
20. Stahl A., Chen J., Sapieha P.; Postnatal weight gain modies severity and functional outcome of oxygeninduced proliferative retinopathy. Am J Pathol. 2010; 177: 2715-2723.
21. Connor K M., Krah N M., Dennison R J.; Quantication of oxygen-induced retinopathy in the mouse: a model of vessel loss, vessel regrowth and pathological angiogenesis. Nat Protoc. 2009; 4: 1565-1573.
22. Stahl A., Connor K M.; Sapieha P Computer-aided quantification of retinal neovascularization. Angiogenesis. 2009; 12: 297-301.
23. Zeng G.; Orientation of endothelial cell division is regulated by VEGF signaling during blood vessel formation. Blood. 2007; 109: 1345-1352.
24. Dejana E.; The role of Wnt signaling in physiological and pathological angiogenesis. Circ Res. 2010; 107: 943-952.
25. Chen J., Stahl A., Krah N M.; Wnt signaling mediates pathological vascular growth in proliferative retinopathy. Circulation. 2011; 124: 1871-1881.
26. Rao T P., Kuvhl M.; An updated overview on Wnt signaling pathways. A prelude for more. Circ Res. 2010; 106: 1798-1806.
27. Mikels A., Nusse R.; Puried Wnt5a protein activates or inhibits β-Catenin-TCF signaling depending on receptor context. PLoS Biol. 2006; 4: 570-582.
28. Tamm E., Ohlmann A.; Norrin in the treatment of diseases associated with an increased TGF-beta activity. European patent application EP20090798920. Sep. 28, 2011.
29. Gammons M V, Rutherford T J, Steinhart Z, Angers S, Bienz M. Essential role of the Dishevelled DEP domain in a Wnt-dependent human-cell-based complementation assay. J Cell Sci. 2016; 129(20):3892-3902.
30. Jernigan K K, Cselenyi C S, Thorne C A, et al. Gbetagamma activates GSK3 to promote LRP6-mediated beta-catenin transcriptional activity. Sci Signal. 2010; 3(121):ra37.
31. Sonmez K., Drenser K., Capone A., Trese M T.; Vitreous levels of stromal cell-derived factor 1 and vascular endothelial growth factor in patients with retinopathy of prematurity. Ophthalmology. 2008; 115: 1065-1070.
32. Robitaille J., Zheng B., Wallace K.; The role of Frizzled-4 mutations in familial exudative vitreoretinopathy and Coats disease. Br J Ophthalmol. 2011; 95: 574-579.
33. Chen Y., Hu Y., Zhou T.; Activation of the Wnt pathway plays a pathogenic role in diabetic retinopathy in humans and animal models. Am J Pathol. 2009; 175: 2676-2685.
34. Wu W C., Drenser K., Trese M T, Capone A., Jr Dailey W.; Retinal phenotype-genotype correlation of pediatric patients expressing mutations in the Norrie disease gene. Arch Ophthalmol. 2007; 125: 225-230.
35. Chen J., Stahl A., Krah N M; Retinal expression of Wnt-pathway mediated genes in low-density lipoprotein receptor-related protein 5 (Lrp5) knockout mice. PLoS One. 2012; 7: e0030203.
36. Olivares A M, Althoff K, Chen G F, et al. Animal Models of Diabetic Retinopathy. Curr Diab Rep. 2017; 17(10):93.
37. Yun N E, Ronca S, Tamura A, Koma T, Seregin A V, Dineley K T, Miller M, Cook R, Shimizu N, Walker A G, Smith J N, Fair J N, Wauquier N, Bockarie B, Khan S H, Makishima T, Paessler S; Animal Model of Sensorineural Hearing Loss Associated with Lassa Virus Infection. Journal of Virology February 2016, 90 (6) 2920-2927.

1. A method of nerve regeneration in a living subject at a situs in a need thereof comprising:
administering to the situs an effective amount of an N-terminus norrin truncate that has a polypeptide N-terminus cleavage relative to a native norrin protein of up to 40 amino acid residues retaining a cysteine-knot motif of the native norrin protein and capable of binding to nerve cells in the situs, or a norrin mutant having at least 85% amino acid identity to SEQ ID NO. 1 and retaining a cysteine-knot motif of the native norrin protein and capable of binding to the nerve cells in the situs; and
allowing sufficient time for said N-terminus norrin truncate or said norrin mutant to selectively up-regulate gene expression of at least one of FZD4, LGR4, LGR5, LGR6, or combinations thereof, and activate a Wnt signaling pathway, to stimulate neurogenesis of the nerve cells in the situs of the living subject.

2. The method of claim 1, wherein said subject is human.

3. The method of claim 1, wherein said subject is one of: a cow, a horse, a sheep, a pig, a goat, a chicken, a cat, a dog, a mouse, a guinea pig, a hamster, a rabbit, or a rat.

4. The method of claim 1, further comprising diagnosing nerve degeneration or nerve damage in the situs of said subject prior to the administering step.

5. The method of claim 1, wherein said N-terminus norrin truncate or said norrin mutant is selected from the group consisting of SEQ ID. NO. 3, 5, 6, 7, 8, 9, 10, 11, 14, and 16.

6. The method of claim 1, wherein said N-terminus norrin truncate or said norrin mutant is selected from the group consisting of SEQ ID. NO. 12, 13, 14, 15, 17, 18, 19, 20, and 21.

7. The method of claim 1, wherein said N-terminus norrin truncate or said norrin mutant is a fragment of SEQ ID. NO. 1 that binds a frizzled-4 receptor of the nerve cells in the ear.

8. The method of claim 1, wherein said N-terminus norrin truncate or said norrin mutant is recombinant.

9. The method of claim 1, wherein the administration is by localized injection to the situs.

10. The method of claim 1, wherein said N-terminus norrin truncate consists of: a polypeptide of SEQ ID. NO. 2.

11. The method of claim 10 further comprising a mutation in at least one position 81-90 relative to SEQ ID NO: 1 that interferes with protease cleavage of the resulting protein.

12. The method of claim 11 wherein the mutation is in at least one of the positions 84, 85, 86, 87, or 88.

13. The method of claim 1, wherein the situs is an inner ear.

14. The method of claim 13 where the nerve cells are mechanosensory.

15. The method of claim 1, wherein the situs is a retina.

16. The method of claim 14 where the nerve cells are retinal ganglia.


SEQUENCE LISTING (21 sequences)

SEQ ID NO: 1 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 2 - protein, 109 aa, Artificial sequence
  Synthetic
KTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 3 - protein, 133 aa, Artificial sequence
  Synthetic
MRKHVLAASFSMLSLLVIMGDTDSKTDSSFIMDSDPRRCMRHHYVDSISHPLYKCSSKMVLLAECEGHCSQASRSEPLVSFSTVLKQPFRSSCHCCRPQTSKLKALRLRCSGGMRLTATYRYILSCHCEECNS

SEQ ID NO: 4 - protein, 133 aa, Artificial sequence
  Synthethic
MRKHVLAASFSMLSLLVIMGDTDSKADSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 5 - protein, 133 aa, Artificial sequence
  Synthetic
MRKHVLAASFSMLSLLVIMGDTDSKTDASFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 6 - protein, 133 aa, Artificial sequence
  Synthetic
MRKHVLAASFSMLSLLVIMGDTDSKTDSAFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 7 - protein, 133 aa, Artificial sequence
  Synthetic
MRKHVLAASFSMLSLLVIMGDTDSKTDSSFIMDSDARRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 8 - protein, 133 aa, Artificial sequence
  Synthetic
MRKHVLAASFSMLSLLVIMGDTDSKTDSSFIMDSDPARCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 9 - protein, 133 aa, Artificial sequence
  Synthetic
MRKHVLAASFSMLSLLVIMGDTDSKTDSSFIMDSDPRACMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 10 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATARYILSCHCEECNS

SEQ ID NO: 11 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYAYILSCHCEECNS

SEQ ID NO: 12 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRAILSCHCEECNS

SEQ ID NO: 13 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCACEECNS

SEQ ID NO: 14 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCAECNS

SEQ ID NO: 15 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEACNS

SEQ ID NO: 16 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLEQPFRSSCHCCRPQTSKLKALRLRCSGGMRLTATYRYILSCHCEECNS

SEQ ID NO: 17 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFESSCHCCRPQTSKLKALRLRCSGGMRLTATYRYILSCHCEECNS

SEQ ID NO: 18 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCEPQTSKLKALRLRCSGGMRLTATYRYILSCHCEECNS

SEQ ID NO: 19 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSELKALRLRCSGGMRLTATYRYILSCHCEECNS

SEQ ID NO: 20 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLEALRLRCSGGMRLTATYRYILSCHCEECNS

SEQ ID NO: 21 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMELTATYRYILSCHCEECNS